(19) United States
(12) Patent Application Publication
(10) Pub. No.: US 2012/0114625 A1
(43) Pub. Date: May 10, 2012

(54) SHELF-STABLE FERMENTED DAIRY PRODUCTS AND METHODS OF MAKING SAME

(75) Inventors: Ana Lucia Wiessel, Spring Lake, MI (US); Allen Bruce Zerlaut, Fremont, MI (US); Frank Karl Welch, Kentwood, MI (US)

(73) Assignee: NESTEC S.A., Vevey (CH)

(21) Appl. No.: 13/265,649

(22) PCT Filed: Apr. 23, 2010
(86) PCT No.: PCT/US10/32263

§ 371 Date: Dec. 21, 2011

(30) Foreign Application Priority Data
Apr. 24, 2009 (US) 61172443

(51) Int. Cl. A23C 13/16; A23C 9/13; A23C 9/133; A23C 9/137; A23C 9/12; A61K 31/07; A61K 31/341; A61K 31/355; A61K 33/04; A61K 31/01; A61K 31/015; A61K 31/122; A61K 38/43; A61K 31/195; A61K 36/00; A61P 39/06; A23C 17/00

(52) U.S. Cl. 424/94.1; 426/580; 426/583; 426/576; 426/577; 426/575; 426/71; 426/72; 426/330.2; 426/334; 426/61; 426/311; 514/725; 514/474; 514/458; 424/702; 514/762; 514/763; 514/690; 514/563; 514/22

(57) ABSTRACT

Shelf-stable fermented dairy products and methods of making the shelf-stable fermented dairy products are provided. The shelf-stable fermented dairy products can be shelf-stable with improved taste, viscosity and texture profiles. In a general embodiment, the present disclosure provides a shelf-stable fermented dairy product including a shelf-stable fermented dairy component, a stabilizer, and a puree composition. The shelf-stable fermented dairy component can be, for example, yogurt, sour cream, buttermilk or a combination thereof.

### PRIORITY CLAIM

This application claims priority to and the benefit of U.S. Provisional Patent Application No 61/172,443 tiled Apr. 24, 2009, the entire content of which is expressly incorporated herein by reference.

### BACKGROUND

The present disclosure generally relates to health and nutrition. More specifically, the present disclosure relates to shelf-stable fermented dairy products and methods of making the shelf-stable fermented dairy products.

There are many refrigerated food products currently on the market. Refrigeration is the process of cooling or freezing the food product to lower temperatures so as to extend the life of the food product. During storage, bacteria within food products can cause the food product to spoil over time. By refrigerating, a food product can be maintained without spoiling for extended periods of time such as weeks or months. Typical food products requiring refrigeration include meat and dairy products including fermented dairy products such as yogurt. However, food products that require refrigeration are generally more costly to store than non-refrigerated foods due to the energy costs associated with refrigeration or freezing.

Shelf-stable foods are foods that would normally he stored refrigerated but have been processed so that they can be safely stored at room or ambient temperature for long shelf life. Various food preservation and packaging techniques are used to extend a foods shelf life. Some of these techniques include decreasing the amount of available water in a food product, increasing its acidity, or irradiating or otherwise sterilizing the food product and then sealing it in an air-tight container. For some foods alternative ingredients can be used. However, different types of food products each required specific techniques to increase the food's shelf life without unacceptably changing its taste or texture.

A fermented dairy product such as yogurt is very susceptible to protein coagulation when heated following the fermentation process. Furthermore, a fermented dairy product introduces a multitude of challenges in maintaining shelf-stability while providing the appropriate taste and texture profiles. Therefore, there is a need for a shelf-stable fermented dairy product that is appealing to a consumer and does not need to be refrigerated.

### SUMMARY

Shelf-stable fermented dairy products and methods of making the shelf-stable fermented dairy products are provided. In a general embodiment, the present disclosure provides a shelf-stable fermented dairy product including a fermented dairy component, a stabilizer, and a puree composition.

In an embodiment of the method, the shelf-stable fermented dairy product has a flavor liking score of at least 5 based on a 9-point hedonic scale of a quantitative central location test. The shelf-stable fermented dairy product can have a sweetness liking score of at least 5 based on a 9-point hedonic scale of a quantitative central location test. The shelf-stable fermented dairy product can have a tartness liking score of at least 5 based on a 9-point hedonic scale of a quantitative central location test. In addition, the shelf-stable fermented dairy product can have a texture liking score of at least 5 based on a 9-point hedonic scale of a quantitative central location test.

In an embodiment of the method, adding the stabilizer to the fermented dairy component under shear comprises stabilizing proteins in the fermented dairy component by coating with the stabilizer. The fermented dairy mixture can be heated to a temperature above 200° F. In addition, the method can be performed under aseptic conditions.

An advantage of the present disclosure is to provide an improved shelf-stable fermented dairy product that is shelf-stable for at least 3 months or longer.

Yet another advantage of the present disclosure is to provide an improved method of making a shelf-stable fermented dairy product.

Still another advantage of the present disclosure is to provide a commercially sterile product that is not grainy and maintains this characteristic over the shelf life of the product.

Another advantage of the present disclosure is to provide a method for making shelf-stable fermented dairy products that is easily adaptable to commercial processes typically in place for heat processed dairy-based products (e.g., such a pudding).

Yet another advantage of the present disclosure is to provide a method for making shelf-stable fermented dairy products having the ability to add a variety of other ingredients to the shelf-stable fermented dairy product without impacting the finished product stability as it relates to the protein matrix of the shelf-stable fermented dairy product.

Additional features and advantages are described herein, and will be apparent from the following Detailed Description.

### DETAILED DESCRIPTION

Shelf-stable fermented dairy products and methods of making the shelf-stable fermented dairy products are provided. The shelf-stable fermented dairy products can be shelf-stable with developmentally appropriate textures and taste profiles. In a general embodiment, the present disclosure provides a shelf-stable fermented dairy product including a fermented dairy component, a physical or chemical stabilizer, and a puree composition. The fermented dairy component can be, for example, dehydrated or fresh yogurt, sour cream, buttermilk, kefir, cheese, or a combination thereof. Other suitable shelf-stable fermented dairy components can also be used to make the shelf-stable fermented dairy products in embodiments of the present disclosure.

As used herein, the term “shelf-stable” means capable of being stored at room temperature (e.g., about 20° C. to about 25° C.) for long periods (e.g., more than 3 months) without becoming spoiled or rotten. Typical fermented dairy products normally need to be stored refrigerated, but the shelf-stable fermented dairy products in embodiments of the present disclosure have been processed so that they can be safely stored in a sealed container at room or ambient temperature for a usefully long shelf life without unacceptably changing their taste or texture. The fermented dairy product produced can be shelf-stable, for example, for more than 3 months, 6 months, 12 months. 18 months, etc.

In an embodiment, the shelf-stable fermented dairy product of the present invention has a taste and flavor profile that yields a liking score from a sensory perspective that is significantly higher than other shelf stable dairy compositions and refrigerated dairy compositions (e.g., obtains or receives from a consumer) a flavor liking score of at least 5, 6, 7, 8 or 9 based on a 9-point hedonic scale of a quantitative central location test. The 9-point hedonic scale is one of the most widely used scale for measuring food acceptability. For example, the 9-point hedonic scale assigns points 1-9 based on user preferences for a food product as follows: Like Extremely—9; Like Very Much—8; Like Moderately—7; Like Slightly—6; Neither Like nor Dislike—5; Dislike Slightly—4; Dislike Moderately—3; Dislike Very Much—2; and Dislike Extremely—1.

Central location tests are product marketing tests performed in controlled environments, contrary to home-user tests, which take place where the products would actually be used. Central location tests can be conducted in a premises such as a room in a shopping mall. Consumers can be recruited to participate in a research product on the shopping mall and the research can be conducted and completed at that time. The consumers can be children or adults. The number of consumers can vary depending on the statistical analysis performed. It should be appreciated that the number of consumers should be enough to provide a statistically relevant test.

The shelf-stable fermented dairy product can have a score of at least 5, 6, 7, 8 or 9 for other characteristics based on a 9-point hedonic scale of a quantitative central location test. For example, the characteristics can include appearance liking, color liking, flavor liking, fruit flavor liking, sweetness liking, tartness liking, texture liking or consistency

In an embodiment the stabilizer is a physical or chemical stabilizer and is a hydrocolloid or a high gelling whey protein, concentrate. The hydrocolloid can be pectin, gelatin, carrageenan, agar, acacia gum, sodium alginate, xanthan gum, locust bean gum, carboxymethyl cellulose (CMC) or a combination thereof. The stabilizer can range from about 0.001% to about 10% by weight, preferably from about 0.01% to 5% and most preferably from about 0.2% to about 0.5%.

In an embodiment, the shelf-stable fermented daisy product has a pH ranging from about 3.8 to about 4.3, preferably from about 3.9 to 4.3 and most preferably about 4.1 to 4.3.

The present invention offers a surprisingly significant difference and preference in viscosity and texture as seen in Tables I—below. Viscosity is measured using a Brookfield RV #6 Spindle at 5 RPM, 10 seconds and ranges from about at least 20000 centipoise, preferably from about 30000 centipoise to about 70000 centipoise and most preferably about 35000 centipoise to about 60000 centipoise. Texture is measured using a TMS-Pro Texture Analyzer-Serial #07-1066-08 and ranges from about 3.000 Newtons to about 5.000, preferably from about 3.200 to about 4.800 and most preferably from about 3.400 to about 4.500.

In a comparative analysis of flavored yogurts of the present invention (A) with yogurts of similar flavor in another shelf stable yogurt product (B) and a refrigerated yogurt product (C), the results showed a statistically significant difference between the viscosity and texture of the present invention and the two other products, as detailed in Tables 1-4 below.

TABLE 1

| | Product | Viscosity | stdev | Texture | stdev |
|---|---|---|---|---|---|
| Straw- | A- Strawberry | 55552 | 1161 | 4.3950 | 0.1605 |
| berry | B- Shelf stable Strawberry | 14120 | 1072 | 1.7822 | 0.0621 |
| | C- Refrigerated Strawberry | 17240 | 1218 | 3.3441 | 0.1300 |
| Banana | A- Banana | 45416 | 1253 | 3.4339 | 0.1135 |
| | B- Shelf stable Banana | 16912 | 1398 | 1.9781 | 0.0816 |
| | C- Refrigerated Banana | 14928 | 1026 | 2.9344 | 0.1307 |
| Pear | A- Pear | 53976 | 3047 | 3.8363 | 0.1618 |
| | B- Shelf stable Pear | 17224 | 1934 | 2.2267 | 0.2410 |
| | C- Refrigerated Pear | 15200 | 1570 | 2.9463 | 0.2703 |
| Peach | A- Peach | 38064 | 1833 | 3.4337 | 0.1332 |
| | B- Refrigerated Peach | 16800 | 2006 | 2.9830 | 0.2113 |

TABLE 4

| Brand | | |
|---|---|---|
| A | B | C |

Texture-Strawberry

| | | | |
|---|---|---|---|
| Texture | 4.40 BC | 1.78 | 3.3 B |

Texture-Banana

| | | | |
|---|---|---|---|
| Texture | 3.43 BC | 1.98 | 2.93 B |

Texture-Pear

| | | | |
|---|---|---|---|
| Texture | 3.84 BC | 2.23 | 2.95 B |

Texture-Peach

| | | |
|---|---|---|
| Texture | 343 B | 2.98 |

Viscosity-Strawberry

| | | | |
|---|---|---|---|
| Viscosity | 55552 BC | 14120 | 17240 B |

Viscosity-Banana

| | | | |
|---|---|---|---|
| Viscosity | 45416 BC | 16912 C | 14928 |

Viscosity-Pear

| | | | |
|---|---|---|---|
| Viscosity | 53976 BC | 17224 C | 15200 |

Viscosity-Peach

| | | |
|---|---|---|
| Viscosity | 38064 B | 16800 |

In the present invention, sensory tests were conducted by trained sensory panelists with a Descriptive Analysis using a 100 point unstructured Line Scale. The results of the sensory analysis of the texture are provided in Table 5 below.

The shelf-stable fermented dairy product can include also include acidulants including but limited to lactic acid, malic acid, citric acid, tartaric acid, phosphoric acid, glocono delta lactone in an amount of about 0.01% to about 2% by weight, preferably from about 0.1-1% by weight.

In an embodiment, the composition of the present invention can include sugar in an amount up to about 20% by weight, preferably from about 3% to 15% by weight, and most preferably from about 5% to about 10% by weight. The shelf-stable fermented dairy product can also be sugar free and include sugarless sweeteners such as mattitol, mannitol, xylitol, hydrogenated starch hydrolysates, sorbitol, lactitol, erythritol and the like, alone or in combination.

High intensity artificial or natural sweeteners can also be used in the shelf-stable fermented dairy product. Preferred sweeteners include, but are not limited to sucralose, aspartame, salts of acesulfame, alitame, saccharin and its salts, cyclamic acid and its salts, glycyrrhizin, stevioside, dihydrochalcones, thaumatin, monellin, and the like, alone or in combination.

In an embodiment, the puree composition includes a pureed fruit including but not limited to apple, orange, pear, peach, strawberry, banana, cherry, pineapple, kiwi, grape, blueberry, raspberry, mango, guava, cranberry, blackberry or a combination thereof. The fruit can be present in an amount ranging from about 0% to about 80% by weight, preferably from about 3% to about 20% by weight and most preferably from about 5% to about 10% by weight, Flavor components in general can range from about 0% to about 10%, preferably from about 0.001% to about 5% and most preferably from about 0.1% to about 4% by weight.

In an embodiment, the composition of the present invention can include a vegetable ingredient selected from the group including but not limited to sweet potatoes, carrots, peas, green beans and squash.

In an embodiment, the shelf-stable fermented dairy product further includes one or more prebiotics. As used herein, a prebiotic is a selectively fermented ingredient that allows specific changes, both in the composition and/or activity in the gastrointestinal microflora, that confers benefits upon host well-being and health. Non-limiting examples of prebiotics include fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols or a combination thereof.

In an embodiment, the shelf-stable fermented dairy product further includes one or more probiotics. As used herein, probiotics are defined as microorganisms (e.g., live) that could confer health benefits on the host when administered in adequate amounts. Non-limiting examples of probiotics include Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof.

In another embodiment, the shelf-stable fermented dairy product further includes one or more amino acids. Non-limiting examples of amino acids include Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Serine, Tyrosine, Arginine, Histidine or a combination thereof.

In an embodiment, the shelf-stable fermented dairy product further includes one or more symbiotics, phytonutrients, antioxidants, vitamins and/or minerals. As used herein, a symbiotic is a supplement that contains both a prebiotic and a probiotic that work together to improve the microflora of the intestine. Non-limiting examples of phytonutrients include quercetin, curcumin and limonin. Antioxidants are molecules capable of slowing or preventing the oxidation of other molecules. Non-limiting examples of antioxidants include vitamin A, carotenoids, vitamin C, vitamin E, selenium, flavonoids, polyphenols, lycopene, lutein, lignan, coenzyme Q10 (“CoQ10”) and glutathione.

Non-limiting examples of vitamins may include Vitamins A, B-complex (such as B-1, B-2, 8-6 and B-12). C, D, E and K, niacin and acid vitamins such as pantothenic acid and folic acid and biotin. Non-limiting examples of minerals may include calcium, iron, zinc, magnesium, iodine, copper, phosphorus, manganese, potassium, chromium, molybdenum, selenium, nickel, tin, silicon, vanadium and boron.

In an alternative embodiment the present disclosure provides a method of making a shelf-stable fermented dairy product. The method comprises adding a physical or chemical stabilizer to a fermented dairy component under shear to create a shelf-stable fermented dairy mixture under a temperature range from 33-65 F at a blending range from 10 to 1000 rpm, preferably from about 50 to 500 rpm and most preferably from about 100 to about 300 rpm., homogenizing the fermented dairy mixture under a temperature range of from about 33 F to about 165 F, preferably about 33 F to about 100 F and most preferably from about 33 F to about 60 F and in a single or dual stage homogenizer with pressure range from about 500 psi to about 4000 psi, preferably from about 500 psi to about 3000 psi, and most preferably from about 500 psi to about 1500 psi, adding a puree composition to the fermented dairy mixture under a temperature range from about 33 F to about 165 F at blending range from 10 to 1000 rpm, and heat processing the shelf-stable fermented dairy mixture to render the shelf-stable fermented dairy mixture commercially sterile to form the shelf-stable fermented dairy product in a range of from about 10 seconds to about 40 minutes, at the temperature range of about 185 F to about 240 F. The method can be performed under aseptic conditions.

The present method unexpectedly creates an improved shelf stable dairy product with improved taste, viscosity and texture. Specifically, refrigerated dairy products coagulate over time and temperature and need to be controlled to obtain the correct viscosity for the end product. High sheer and heat are not necessary and not preferred in the prior art methods since natural proteins create viscosity and thickness which coagulate and form a matrix to build the texture and viscosity of the final product. The method of the present invention surprisingly provided improved viscosity, texture and taste. While viscosity alone may be adjustable in the prior art refrigerated methods, the combination of the viscosity and texture of the present invention provides a surprisingly improved and preferred composition.

The first part of the method involves “stabilizing” protein in the shelf-stable fermented dairy component by coating it with a suitable hydrocolloid pectin) or a high gelling whey protein concentrate followed by homogenization of the shelf-stable fermented dairy mixture. This allows the shelf-stable fermented dairy mixture to be heated to sterilization temperatures (e.g., above 185° F.) without coagulating the protein thereby resulting in a smooth textured fermented dairy product.

In an embodiment of the method, one or more thickeners can include but are not limited to physically or chemically modified flours and/or starches from sources such as rice, wheat, oat, barley, tapioca, quinoa, rye, amaranth, corn, or potatoe. Flavors and/or colors are added to the fermented dairy mixture before the heat processing. The shelf-stable fermented dairy component can be yogurt, sour cream, buttermilk or a combination thereof.

Embodiments of the present disclosure advantageously provide the capability to produce a commercially sterile, shelf-stable fermented dairy product that is not grainy while maintaining this characteristic over the shelf life of the product. Available commercial processes typically in place for heat processed, dairy-based products (e.g., such a pudding) can be used to make the shelf-stable fermented dairy products. Various ingredients can be added to the shelf-stable fermented dairy products during the manufacturing process without impacting finished product stability as it relates to the protein matrix of the shelf-stable fermented dairy products.

### EXAMPLES

By way of example and not limitation, the following examples are illustrative of various embodiments of the present disclosure. The formulations below are provided for exemplification and they can be modified by the skilled artisan to the necessary extent, depending on the special features that are looked for.

### Example 1

### Yogurt Blends—Banana

| Material Name | Percent |
|---|---|
| Full Fat Yogurt, Refrigerated | 85.06 |
| Sugar | 5.54 |
| Banana Puree, Deseeded | 5.00 |
| Tapioca Starch Physically Treated | 3.50 |
| Flavor, Banana | 0.54 |
| Pectin | 0.35 |
| Color Turmeric | 0.003 |
| Citric Acid | 0.01 |

### Yogurt Blends—Peach

| Material Name | Percent |
|---|---|
| Full Fat Yogurt, Refrigerated | 85.15 |
| Sugar | 5.55 |
| Peach Puree Concentrate | 3.04 |
| Water to reconstitute puree | 1.86 |
| Tapioca Starch Physically Treated | 3.50 |
| Flavor, Peach | 0.54 |
| Pectin | 0.35 |
| Color, Annatto | 0.01 |
| Citric Acid | 0.01 |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A shelf-stable fermented dairy product comprising a fermented dairy component, a stabilizer, and a puree composition.

2. The shelf-stable fermented dairy product of claim 1, wherein the shelf-stable fermented dairy product has a flavor liking score of at least 5 based on a 9-point hedonic scale of a quantitative central location test.

3. The shelf-stable fermented dairy product of claim 1, wherein the shelf-stable fermented dairy product has a sweetness liking score of at least 5 based on a 9-point hedonic scale of a quantitative central location test.

4. The shelf-stable fermented dairy product of claim 1, wherein the shelf-stable fermented dairy product has a tartness liking score of at least 5 based on a 9-point hedonic scale of a quantitative central location test.

5. The shelf-stable fermented dairy product of claim 1, wherein the shelf-stable fermented dairy product has a texture liking score of at least 5 based on a 9-point hedonic scale of a quantitative central location test.

6. The shelf-stable fermented dairy product of claim 1, wherein the shelf-stable fermented dairy component is selected from the group consisting of yogurt, sour cream, buttermilk and combinations thereof.

7. The shelf-stable fermented dairy product of claim 1, wherein, the stabilizer is physical or chemical stabilizer and is selected from the group consisting of a hydrocolloid or a high gelling whey protein concentrate.

8. The shelf-stable fermented dairy product of claim 7, wherein the stabilizer is selected from the group consisting of pectin, gelatin, carrageenan, agar, acacia gum, sodium alginate, xanthan gum, locust bean gum, carboxymethyl cellulose, high gelling whey protein concentrate and combinations thereof.

9. The shelf-stable fermented dairy product of claim 1, wherein the physical stabilizer ranges from 0.001% to 10% by weight.

10. The shelf-stable fermented dairy product of claim 1 comprising a pH ranging from 3.8 to 4.3.

11. The shelf-stable fermented dairy product of claim 1, wherein the puree composition comprises a pureed fruit selected from the group consisting of apple, orange, pear, peach, strawberry, banana, cherry, pineapple, kiwi, grape, blueberry, raspberry, mango, guava, cranberry, blackberry and combinations thereof.

12. The shelf-stable fermented dairy product of claim 1 further comprising a prebiotic.

13. The shelf-stable fermented dairy product of claim 12, wherein the prebiotic is selected from the group consisting of partially hydrolyzed guar gum, fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols and combinations thereof.

14. The shelf-stable fermented dairy product of claim 1 further comprising a probiotic.

15. The shelf-stable fermented dairy product of claim 14, wherein the probiotic is selected from the group consisting of Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Aerococcus, Oenococcus, Lactobacillus and combinations thereof.

16. The shelf-stable fermented dairy product of claim 1 further comprising a component selected from the group consisting of symbiotics, phytonutrients and combinations thereof.

17. The shelf-stable fermented dairy product of claim 1 further comprising an amino acid.

18. The shelf-stable fermented dairy product of claim 17, wherein the amino acid is selected from the group consisting of Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Serine, Tyrosine, Arginine, Histidine and combinations thereof.

19. The shelf-stable fermented dairy product of claim 1 further comprising an antioxidant.

20. The shelf-stable fermented dairy product of claim 1 further comprising a vitamin.

21. The shelf-stable fermented dairy product of claim 1 further comprising a mineral.

22. A method of making a shelf-stable fermented dairy product, the method comprising;
adding a stabilizer to a fermented dairy component under shear to create a shelf-stable fermented dairy mixture;
homogenizing the fermented dairy mixture;
adding a puree composition to the fermented dairy mixture; and
heat processing the fermented dairy mixture to render the fermented dairy mixture commercially sterile to form the shelf-stable fermented dairy product.

23. The method of claim 22, wherein adding the stabilizer to the fermented dairy component under shear comprises stabilizing proteins in the fermented dairy component by coating with the physical stabilizer.

24. The method of claim 22, wherein the fermented dairy mixture is heated to a temperature above 185° F.

25. The method of claim 22, wherein the method is performed under aseptic conditions.

26. The method of claim 22, wherein at least one of thickeners, flavors, sweeteners, acidulants and colors is added to the mixture before the heat processing.

27. The method of claim 22, wherein the shelf-stable fermented dairy product has a flavor liking score of at least 5 based on a 9-point hedonic scale of a quantitative central location test.

28. The method of claim 22, wherein the shelf-stable fermented dairy product has a sweetness liking score of at least 5 based on a 9-paint hedonic scale of a quantitative central location test.

29. The method of claim 22, wherein the shelf-stable fermented dairy product has a tartness liking score of at least 3 based on a 9-point hedonic scale of a quantitative central location test.

30. The method of claim 22, wherein the shelf-stable fermented dairy product has a texture liking score of at least 5 based on a 9-point hedonic scale of a quantitative central location test.

31. The method of claim 22, wherein the fermented dry or fresh dairy component is selected from the group consisting of yogurt, sour cream, buttermilk, kefir cheese and combinations thereof.

32. The method of claim 22, wherein the stabilizer is a physical or chemical stabilizer.

33. The method of claim 32, wherein the stabilizer is selected from the group consisting of pectin, gelatin, carrageenan, agar, acacia gum, sodium alginate, xanthan gum, locust bean gum, carboxymethyl cellulose, high gelling whey protein concentrate and combinations thereof.

34. The method of claim 22, wherein the stabilizer ranges from 0.001% to 10% by weight.

35. The method of claim 22, wherein the shelf-stable fermented dairy product comprises a pH ranging from about 3.8 to about 4.3.

36. The method of claim 22, wherein the puree composition comprises a pureed fruit selected from the group consisting of apple, orange, pear, peach, strawberry, banana, cherry, pineapple, kiwi, grape, blueberry, raspberry, mango, guava, cranberry, blackberry and combinations thereof.

37. The method of claim 22, wherein the shelf-stable fermented dairy product further comprises a prebiotic.

38. The method of claim 22, wherein the prebiotic is selected from the group consisting of partially hydrolyzed guar gum, fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols and combinations thereof.

39. The method of claim 22, wherein the shelf-stable fermented dairy product further comprises a probiotic.

40. The method of claim 39, wherein the probiotic is selected from the group consisting of Saccharomyces, Debaromyces Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus Pediococcus, Micrococcus, Leuconostoc, Weissella, Aerococcus, Oenococcus, Lactobacillus and combinations thereof.

41. The method of claim 22, wherein the shelf-stable fermented dairy product further comprises a component selected from the group consisting of symbiotics, phytonutrients and combinations thereof.

42. The method of claim 22, wherein the shelf-stable fermented dairy product further comprises an amino acid.

43. The method of claim 42, wherein the amino acid is selected from the group consisting of Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Serine, Tyrosine, Arginine, Histidine and combinations thereof.

44. The method of claim 22, wherein the shelf-stable fermented dairy product further comprises an antioxidant.

45. The method of claim 22, wherein the shelf-stable fermented dairy product further comprises a vitamin.

46. The method of claim 22, wherein the shelf-stable fermented dairy product further comprises a mineral.
